(19) United States
(12) Patent Application Publication
(10) Pub. No.: US 2008/0108129 A1
(43) Pub. Date: May 8, 2008

(54) Method for Obtaining Modified Proteins and Viruses with Intact Native Binding Site

(75) Inventors: Jacob Pitcovski, Korazim (IL); Jacob Vaya, Mizpe Amuka (IL)

Correspondence Address: BROWDY AND NEIMARK, P.L.L.C.;624 NINTH STREET, NW, SUITE 300, WASHINGTON, DC, 20001-5303, US

(73) Assignee: Gavish-Galillee Bio Applications Ltd., Kiryat Shmona (IL)

(21) Appl. No.: 11/813,273

(22) PCT Filed: Dec. 29, 2005
(86) PCT No.: PCT/IL05/01398

§ 371 Date: Jul. 2, 2007

(30) Foreign Application Priority Data
Dec. 30, 2004 (IL) 166049

(51) Int. Cl. C12N 7/06; C07K 1/00; C07K 16/18

(52) U.S. Cl. 435/238; 530/402; 530/387.1; 530/391.1

(57) ABSTRACT

Methods for obtaining modified proteins or virus with an intact native binding site and decreased antigenicity and modified proteins or virus obtainable by said methods are provided. The methods of protein or virus modification comprise masking with non-immunogenic molecules the protein or the virus surface, except for the protein or the virus binding site. Examples of modified proteins or virus that can be modified in accordance to the methods include polyclonal or monoclonal antibodies, modified replication-defective virus, hormones, and enterotoxins.

### FIELD OF THE INVENTION

The present invention relates to methods for obtaining modified proteins, e.g. antibodies, and viruses with an intact native binding site and decreased antigenicity, and to the modified proteins, e.g. antibodies, and viruses thus obtained.

ABBREVIATIONS: Ad: Adenovirus; ADA: adenosine deaminase; AM: attachment molecule; CMD: carboxymethyl dextran; CT: cholera toxin of Vibrio cholera; DMAP: 4-(dimethylamino) pyridine; EDC: 1-ethyl-3-(3-dimethylamino-propyl) carbodiimide; EDS: Egg-drop syndrome; GM-CSF: granulocyte-macrophage colony-stimulating factor; HA: hemagglutination activity; hGH: human growth hormone; HRP: horseradish peroxidase; IFN-α 2b: interferon-α 2b; IL-2: interleukin-2; LT: enterotoxin of Escherichia coli; PEG: polyethylene glycol; PTH: parathyroid hormone; TNF-α: tumor necrosis factor alpha; pTSA: p-toluenesulfonic acid.

### BACKGROUND OF THE INVENTION

A variety of protein-masking procedures are known for changing protein properties. The purposes of such modification include: (i) reduction of protein immunogenicity, for example to reduce allergy to food such as allergy to β-lactoglobulin, the major allergen in cow's milk, or to reduce immunogenicity of therapeutic proteins that elicit antibodies when injected, for example, human rIL-2, tested as anticancer agent; (ii) change of the protein's surface properties by forming barriers between the specific protein and its surrounding, for example change of protein adhesion to cells, thus preventing thrombosis and/or reducing platelet deposition on tissue surface, or change in the solubility of the protein when conjugated and of its circulation in vivo; and (iii) increase of the plasma half-life of a protein drug.

The main procedure used for reducing immunogenicity of proteins is conjugation with polymers such as hydroxylated polyethers or polysaccharides. The major hydroxylated polyether used for this purpose is polyethylene glycol (PEG).

The conjugation with PEG, also known as “pegylation” or “PEGylation”, is a technology for modifying the physical and chemical properties of molecules by chemically attaching functionalized PEG polymer chains to drug substances, including proteins, peptides, enzymes and other bioactive molecules. Pegylation is primarily used to prolong the action of therapeutic proteins. When attached to a drug, it can modify its biological profile depending on the site of attachment and molecular weight of the PEG molecule used. Peptide and protein PEGylation is usually undertaken to improve the biopharmaceutical properties of these drugs. Pegylation also bestows several other clinically useful properties to the parent molecule that include enhanced solubility, reduced immunogenicity, resistance to proteolysis, and reduced toxicity. PEG is considered as a non-adhesive biomaterial due to its ability to resist protein adsorption.

Pegylation has been used to modify a variety of proteins with clinical applications, including adenosine deaminase (ADA), L-asparaginase, interleukin-2 (IL-2), interferon-α 2b (IFN-α 2b), granulocyte-macrophage colony-stimulating factor (GM-CSF), tumor necrosis factor alpha (TNF-α), and human growth hormone (hGH). These pegylated proteins have improved pharmacological properties and, in some cases, have been shown to be more potent than the native protein.

Pegylation technology has been applied also to antibodies and antibody fragments. PEG has been predominantly used to reduce the immunogenicity and increase the circulating half-lives of antibodies. It may also have a beneficial effect on the use of antibodies in certain clinical settings such as tumor targeting.

U.S. Pat. No. 4,732,863 discloses PEG-modified antibodies characterized by reduced immunogenicity, decreased binding capacity for Fc cell surface receptors, and full antigen-binding activity, useful in a variety of immunologically-based diagnostic and therapeutic procedures.

In the case of antibody fragments, PEGylation has not been shown to extend serum half-life to useful levels, and Pedley et al. (1994) reported studies characterizing blood clearance and tissue uptake of certain anti-tumor antigen antibodies or antibody fragments derivatized with low molecular weight (5 kD) PEG. Koumenis et al. (2000) reported that low molecular weight (5 or 10 kD) PEG attached to epsilon-amino groups in the hinge region of a Fab′ fragment reduced clearance compared to the parental Fab′ molecule. U.S. Pat. No. 6,468,532 discloses such pegylated anti-IL-8 monoclonal antibodies fragments.

Polysaccharides have also been used for protein modification. The major polysaccharide used in this context is dextran or modified dextran, e.g. carboxymethyl dextran (CMD), of various molecular weights.

Kobayashi et al. (2001) disclose conjugates of beta-lactoglobulin with carboxymethyl dextran showing reduced immunogenicity. Mehvar (2003) reviews methods of delivery of therapeutic agents using polysaccharides such as dextran, pullulan and mannan (a mannose polymer), and mentions that polysaccharide-protein conjugates increase the duration of effect and decrease the immunogenicity of the proteins.

U.S. Pat. No. 5,698,405 discloses a method of reducing immunogenicity of avidin or of the therapeutic agent moiety of a conjugate, e.g. a toxin, by coupling avidin or said agent with a carbohydrate polymer such as polysaccharides, e.g. dextran, or to polyol groups, e.g. PEG.

Replication-defective recombinant adenovirus vectors are under development for a wide variety of gene therapy indications. Recombinant adenoviruses are presently the most efficient in vivo gene transfer system available. A potential limiting factor associated with adenovirus gene therapy requiring repeated treatments is the development of a humoral immune response to the vector by the host. O'Riordan et al. (1999) have shown that covalent attachment of PEG to the surface of the adenovirus can be achieved with retention of infectivity and that the PEG-modified adenovirus can be protected from antibodies neutralization.

Suppression of human immunologic response to foreign antibodies without destruction of antibody activity has been previously accomplished by enzymatic digestion of the antibody to cleave the Fc fragment of the molecule. The product fragments retain binding capacity for antigen and can be coupled with a variety of chemicals to provide complexes of low immunogenicity. Protease digestion of antibodies is, however, a slow process with low yields, requiring separation of the product fragments.

A PEG 6000 derivative of rabbit antihuman serum albumin was prepared employing a cyanuric chloride coupling procedure. While the product exhibited reduced immunogenicity, loss of avidity for antigen was obtained. A similar result was obtained in a related study, wherein it was concluded that PEG-modification of Ig mediated with cyanuric chloride destroyed antibody activity.

It would be very desirable to provide proteins and viruses with an intact native binding site and decreased antigenicity for different therapeutic and diagnostic purposes.

### SUMMARY OF THE INVENTION

The present invention provides a method for obtaining a modified protein or virus with an intact native binding site and decreased antigenicity by masking with non-immunogenic molecules the protein or virus surface, except for the protein or the virus binding site.

The modified protein is preferably a modified polyclonal or monoclonal antibody and the non-immunogenic molecules are preferably small molecules such as monosaccharides and/or fatty acids, but also polymeric molecules are encompassed by the invention.

The invention further relates to modified proteins, e.g. modified antibodies, and modified viruses obtainable by the methods of the present invention, and to their various applications in therapy and diagnostics.

### BRIEF DESCRIPTION OF THE FIGURES

FIGS. 1A-1B show, respectively, Coomassie blue-stained SDS-PAGE gel of mannose-masked chicken IgY by three different procedures, and Western blot analysis of the mannose-masked chicken IgY using rabbit anti-IgY antibodies. Lane 1: chicken IgY masked with mannose+EDC+NaBH₃CN, sample not boiled prior to loading; Lane 2: chicken IgY masked with mannose+EDC+NaBH₃CN, sample boiled prior to loading; Lane 3: chicken IgY masked with mannose+EDC+pTSA, sample not boiled prior to loading; Lane 4: chicken IgY masked with mannose+EDC+pTSA, sample boiled prior to loading; Lane 5: chicken IgY masked with mannose+EDC, sample not boiled prior to loading; Lane 6: chicken IgY masked with mannose+EDC, sample boiled prior to loading; Lane 7, non-masked chicken IgY, sample not boiled prior to loading; Lane 8: non-masked chicken IgY, sample boiled prior to loading; Lane 9: molecular weight markers.

FIG. 2A-2B show (A) ELISA titration of anti-cow IgG antibodies in sera of chicken following immunization with cow IgG antibodies masked with: mannose+EDC+pTSA (2), mannose+EDC+pTSA+oleic acid and dialysis (3), mannose+EDC+pTSA+oleic acid and no dialysis (4), and oleic acid+EDC+pTSA (5). (B) ELISA titration of anti-human IgG antibodies in sera of chicken before (1) or following immunization with human IgG antibodies masked with mannose+EDC+pTSA+oleic acid and dialysis (2) or immunization with unmasked IgG antibodies (3).

FIG. 3 shows dot blot analysis using anti-EDS antibodies for the detection of adenovirus incubated with anti-knob antibody, following masking with mannose+EDC+pTSA.

### DETAILED DESCRIPTION OF THE INVENTION

According to one aspect of the present invention, a method is provided for preparation of modified proteins and viruses with an intact native binding site and decreased antigenicity by masking with non-immunogenic molecules the protein or the virus surface, except for the protein or the virus binding site.

In one preferred embodiment, the molecules attached covalently to the protein or the virus surface are small molecules, more preferably monosaccharides or fatty acids, or both.

In contrast to major published studies wherein proteins were masked using synthetic polymers or polysaccharides as masking agents, in one preferred embodiment of the present invention small molecules, preferably endogenous molecules, are used for masking the proteins or the viruses. The small molecules include, without being limited to, monosaccharides and/or fatty acids. The advantages of using small molecules reside on their wide diversity and thus the type of masking agents, preferably endogenous small molecules, which can be used is practically unlimited. Each one of these masking agents will have a different influence on the physical and chemical properties of the masked protein or virus. Thus, according to one embodiment of the invention, the small molecule is selected such as to perform a preferential required binding with a protein or virus surface functional groups. For example, a carboxyl group of a fatty acid molecule can react with the protein free amino group residues of lysine or arginine to form amide groups or with the free hydroxyl or sulfidryl groups of serine, threonine, tyrosine or cysteine to form ester or thio-ester groups. In another example, mannose can react with the protein free carboxylic acid residues of aspartic or glutamic acid to form esters. Such options allow controlling the desired degree of masking by choosing the type of amino acids residues of the protein or the virus surface to be bound, in order to reach the desired and most suitable reduction of the degree of immunogenicity. Moreover, the agent selected for masking the protein or the virus surface may be attached to molecules which target specific cells or tissues, and thus the modified protein, e.g. modified antibody, may be used to deliver this agent to the desired specific target.

In one preferred embodiment of the invention, the small molecules are monosaccharides such as, but not limited to, ketoses or aldoses of 3-6 carbon atoms, preferably aldoses of 5-6 carbon atoms, more preferably manose. Masking of the protein or the virus with mannose can be performed, for example, in two steps: first, the free amino groups on the protein or the virus surface are masked by reaction with the aldehyde moiety of mannose, followed by addition of a coupling reagent such as 1-ethyl-3-(3-dimethylaminopropyl) carbodiimide (EDC), to facilitate esterification of the free carboxyl groups on the protein or the virus surface with the mannose hydroxyl groups. In this way, two functional groups of the amino acid residues of the protein or the virus—amino and carboxyl groups—are protected to a degree that is related to the reaction conditions such as type of solvent used (protic, aprotic, polar, etc), type and amount of esterification catalyst used, e.g., EDC, p-toluenesulfonic acid (pTSA), and/or 4-(dimethylamino) pyridine (DMAP), the ratio between the reactants (protein/monosaccharide), reaction time, etc. The double bond of the imine group of the Schiff's base formed in the first step can be further reduced, for example, with NaH₃BCN, in order to increase the stability of the masked molecule.

The attachment of mannose residues to the protein, preferably antibody, surface, may also manipulate a stronger or directed response of the immune system against the antigen-antibody in the body, following treatment, since the mannose residues will enhance phagocytosis through the mannose receptor on macrophages. As a result of the masking procedure the protein plasma half-life can be modulated.

In another preferred embodiment of the invention, the non-immunogenic small molecules are C₃-C₂₀ fatty acids. The fatty acid may be a saturated C₃-C₂₀ fatty acid such as, but not limited to, capric acid, lauric acid, myristic acid, palmitic acid, stearic acid, or arachidic acid, or an unsaturated C₃-C₂₀ fatty acid such as, but not limited to, palmitoleic acid, oleic acid, vaccenic acid, linoleic acid, γ-linolenic acid, linolenic acid, or arachidonic acid. In one most preferred embodiment, the fatty acid molecule is oleic acid.

In another preferred embodiment of the invention, the protein or the virus surface is masked both with mannose and oleic acid residues.

Masking of the protein or the virus surface with fatty acids is performed by esterification of free hydroxyl groups on the protein or the virus surface.

In one embodiment, the esterification is carried out as a first step in the masking procedure, in the presence of catalysts such as EDC and p-TSA. The esterification can also be carried as a second or third step, after reaction of the protein or the virus with other selected small molecules such as a monosaccharide, e.g., mannose, thus obtaining protein or virus molecules which surface is masked by different types of masking materials. The amount of each one of the masking materials is determined by the desired protein or virus properties. It should be noted that mannose reacts with free amino groups to form Schiff bases and also with free carboxylic groups to form esters. The addition of fatty acids as a third step after the two types of reactions with mannose will lead to the esterification of free hydroxyl or sulfidryl groups.

The attachment of fatty acid residues is suitable when a hydrophobic protein or virus surface is desired. By using unsaturated fatty acids for masking such as oleic, linoleic, linolenic, or arachidonic acid, further modifications are possible such as oxidation of the double bond(s) to form diols or epoxides, addition reactions or electrophilic substitutions, including increasing the masking by forming bridges between two neighbor fatty acid chains.

Further modifications of the protein or virus surface are possible by using any other compound capable of reacting with free amino, carboxyl, hydroxyl or thiol groups. It is also desirable to remove excess of the reagent, e.g. EDC, p-TSA, or excess of the reactants, e.g. mannose, fatty acid, after each reaction step, by dialysis or filtration with appropriate cutoff. These reagents are usually added in excess relative to the protein. Such purification of the masked protein may decrease the immunogenic response when the modified protein is injected to the body.

In another embodiment of the present invention, the non-immunogenic molecules attached covalently to the protein or the virus surface are polymer molecules, such as hydroxylated polyethers, more preferably polyethylene glycol (PEG), or a polysaccharide such as dextran, modified dextran, pullulan or mannan.

When the protein is an antibody, masking with the molecules defined above such as fatty acids, monosaccharides and polysaccharides that still contain free functional groups after binding to the antibody surface, will enable efficient binding of further desired molecules to the antibody surface. For example, toxic molecules can be bound to antibodies targeted to cancer cells, or peptides or proteins such as hormone peptides, e.g. melanocyte-stimulating hormones (melanotropins), which target specific cells or tissues, can be bound to antibodies, for clinical therapy. In one preferred embodiment, the targeted specific cells or tissues are cancerous cells or tissues. The toxic molecules can be also bound to virus or to virus-like particles surface with a specific receptor, which target specific cells.

In one preferred embodiment, the present invention provides a method for obtaining a modified antibody with an intact native binding site and decreased antigenicity, by masking the antibody surface with non-immunogenic molecules, except for the native binding site, said method comprising:

(i) attaching an antigen recognized by said antibody to a surface;

(ii) incubating the antibody with said attached antigen, thus forming an antigen-antibody complex;

(iii) masking the antibody surface by chemically attaching non-immunogenic molecules to the antibody surface in the antigen-antibody complex, thus obtaining an antigen-masked antibody complex; and

(iv) separating the masked antibody from the antigen,

thus obtaining the desired modified antibody masked with the non-immunogenic molecules, said modified antibody exhibiting an intact native binding site and decreased antigenicity as compared with the unmodified antibody.

As used herein, the term “antibodies” refers to polyclonal and monoclonal antibodies of avian, e.g. chicken, and mammals, including humans, and to fragments thereof such as F(ab′)₂ fragments of polyclonal antibodies, and Fab fragments and single-chain Fv fragments of monoclonal antibodies.

One of the problems encountered in the administration of foreign molecules to an organism is their antigenicity. Foreign molecules, e.g., antibodies, that are produced in another species and used for passive vaccination, induce an immune response in the host and cannot be used repeatedly.

One main aim of the present invention is to provide masked IgG antibodies with intact native binding site such that their binding specificity remains unchanged while decreasing or eliminating their antigenicity. The masking of the antibody surface with the non-immunogenic molecules according to the present invention decreases its antigenicity and prevents recognition of the antibody by the immune system.

Modified antibodies according to the present invention enable cross-species vaccination. For example, chicken- or horse-derived polyclonal antibodies against snake toxins can be used for vaccination of humans to treat snakebite envenomation. In another example, murine monoclonal antibodies or human monoclonal antibodies that cause an immune response in a human host may be modified according to the present invention, therefore preventing or eliminating the undesired immune responses. Thus, in one preferred embodiment, the present invention provides a modified antibody, which is not antigenic within the same or heterologous species.

In a further aspect, the present invention provides a modified antibody obtainable by the method of the invention, said antibody being biologically-active, having its native binding site intact and exhibiting decreased immunogenicity, as compared to unmodified antibody. In one preferred embodiment, the modified antibody is an anti-tumor antibody.

In still another aspect, the present invention provides a modified antibody wherein its surface, except for the native binding site, is covered by small molecules covalently linked to functional groups of the antibody molecule, and said small molecules are monosaccharides or fatty acids, or both.

In one embodiment of the present invention, the modified antibodies may be useful for a passive vaccination in humans or animals against bacteria such as group A Streptococcus (GAS), Mycobacteria, Staphylococci, Vibrio, Enterobacter, Enterococcus, Escherichia, Haemophilus, Neisseria, Pseudomonas, Shigella, Serratia, Salmonella, Klebsiella and Yersinia.

In another embodiment, the modified antibodies may be also useful for a passive vaccination in humans or animals against viruses such as Influenza, hepatitis A, hepatitis B, Lyme, and West Nile virus.

The method of the present invention can also be applied to viruses such that the virus will exhibit decreased antigenicity and a native knob-binding site.

Adenovirus (Ad) is a group of nonenveloped double-stranded DNA viruses associated with a range of respiratory, ocular, and gastrointestinal infections. Entry of human Ad into human cells is a stepwise process. The primary event in this sequence is attachment that involves an interaction between the Ad fiber protein and its high-affinity cellular receptor. The Ad type 5 (Ad5) fiber is a homotrimer with each subunit consisting of three domains: the amino-terminal tail that associates with the penton base protein; the shaft, which consists of a motif of approximately 15 residues that is repeated 22 times; and the knob, which interacts with the cellular receptor.

A replication-defective adenovirus vector has been used for efficient delivery of DNA and is applicable in adenovirus-mediated gene delivery in gene targeting and gene therapy.

Thus, in yet a further aspect, the present invention provides a method for obtaining a modified replication-defective virus with a native knob binding site and decreased antigenicity, by masking the virus surface with non-immunogenic molecules, except for the protected knob binding site, said method comprising the steps:

(i) incubating the virus with antibodies to the knob binding site, thus forming a virus-antibody complex;

(ii) masking the virus surface by chemically attaching non-immunogenic molecules to the virus surface in the virus-antibody complex, thus obtaining a masked virus-antibody complex;

(iii) separating the masked virus from the antibody; and

(iv) removing the residual antibody from the solution by ultracentrifugation, thus obtaining the desired modified virus masked with the non-immunogenic molecules said modified virus exhibiting an intact native knob binding site and decreased antigenicity as compared with the unmodified virus.

In one preferred embodiment, said replication defective virus is a retrovirus. In a more preferred embodiment, said virus is an adenovirus, most preferably Ad5. In a further embodiment, the virus is a recombinant virus, which expresses a transgene, e.g. a therapeutic gene for use in gene therapy, or an antigen for use in vaccination.

In still another aspect, the present invention provides a method for obtaining a modified hormone with an intact receptor-binding site, by masking the hormone surface with non-immunogenic molecules, except for the protected receptor-binding site, said method comprising the steps:

(i) incubating the hormone with its receptor, thus forming a hormone-receptor complex;

(ii) masking the hormone surface by chemically attaching non-immunogenic molecules to the hormone surface in the hormone-receptor complex, thus obtaining a masked hormone-receptor complex; and

(iii) separating the masked hormone from the receptor; thus obtaining the desired modified hormone masked with the non-immunogenic molecules, said modified hormone exhibiting an intact receptor binding site and decreased antigenicity as compared with the unmodified hormone.

In still a further aspect, the present invention provides a modified hormone obtainable by the method of the invention described above, said hormone having a native receptor binding site and exhibiting decreased immunogenicity, as compared to the unmodified hormone. In one embodiment, said modified hormone is parathyroid hormone (PTH). In another embodiment, said modified hormone is human growth hormone (hGH) with prolonged half-life in body.

In one embodiment, said modified hormones may be useful for targeting specific cancer cells or tissues which express their receptor such as prostate cancer, ovarian cancer, cervical cancer and uterine cancer, which express growth hormone secretagogue type 1b receptor or androgen receptor.

In another aspect, the present invention provides a method for obtaining a modified enterotoxin, with an intact receptor-binding site, by masking the enterotoxin surface with non-immunogenic molecules, except for the protected receptor-binding site, said method comprising the steps:

(i) incubating an enterotoxin with its receptor, thus forming an enterotoxin-receptor complex;

(ii) masking the enterotoxin surface by chemically attaching non-immunogenic molecules to the enterotoxin surface in the enterotoxin-receptor complex, thus obtaining a masked enterotoxin-receptor complex; and

(iii) separating the masked enterotoxin from the receptor;

thus obtaining the desired modified masked enterotoxin with an intact receptor binding site and decreased antigenicity as compared with the unmodified enterotoxin.

In a further aspect, the present invention provides a modified enterotoxin obtainable by the method of the invention described above, said enterotoxin having a native receptor binding site and exhibiting decreased immunogenicity, as compared to the unmodified enterotoxin. In one embodiment, said modified enterotoxin is enterotoxin of Escherichia coli (LT) with an intact GM1 ganglioside receptor-binding site. In another embodiment, said modified enterotoxin is cholera toxin of Vibrio cholera (CT) with an intact GM1 ganglioside receptor-binding site.

In one embodiment, said modified enterotoxin may be useful for delivery of molecules into cells via oral or skin routes.

The invention will now be illustrated by the following non-limiting Examples.

### EXAMPLES

### Materials and Methods

(i) Coating with D(+)-mannose. Coating of chicken IgY with mannose was performed in two steps:(i) masking the free amino groups on the antibody surface by reaction with the aldehyde moiety of mannose, for 12 hours, at room temperature; and (ii) addition of the coupling reagent EDC, optionally together with p-TSA and/or DMAP, to facilitate esterification reaction between the mannose hydroxyl groups and the free carboxylic acid residues. The solvent used is preferably phosphate-buffer, pH 7. To increase the stability of the reaction product, a reducing agent which can reduce the imine double bond C=N is used, e.g. NaH₃BCN.

(ii) Coating with fatty acids. Masking of chicken IgY or cow IgG by esterification with oleic acid was carried out in the presence of the catalyst EDC and pTSA. The esterification was carried out as a second step, after reaction of the antibody with mannose.

### EXAMPLE 1

### Preparation of Antibody Coated by Mannose

In this preliminary example, cow IgG or chicken IgY was coated with mannose by three different coating procedures: (i) mannose+EDC; (ii) mannose+EDC+pTSA; (iii) mannose+EDC+NaBH₃CN.

(i) Coating with mannose+EDC

D(+)-mannose (10 mg) was added to a 0.5 ml solution of cow IgG or chicken IgY (225 μg/ml solution in PBS 50 mM, pH=7.0), the solution was shaken for 12 hours at room temperature, then 9 mg of EDC was added, and the mixing was continued for additional 12 hours.

(ii) Coating with mannose+EDC+pTSA

D(+)-mannose (10 mg) was added to a 0.5 ml solution of cow IgG or chicken IgY (225 μg/ml solution in PBS 50 mM, pH=7.0), the solution was shaken for 12 hours at room temperature, then 3 mg of p-TSA and 9 mg of EDC were added, and the mixing was continued for additional 12 hours.

(iii) Coating with mannose+EDC+NaBH₃CN

D(+)-mannose (10 mg) was added to a 0.5 ml solution of chicken IgY (1 mg/ml solution in PBS 50 mM, pH=7.0), the solution was shaken for 12 hours at room temperature, then 6 mg of EDC was added, and the mixing was continued for additional 12 hours. NaBH₃CN (5 mg) was then added and the solution was mixed for another 12 hours.

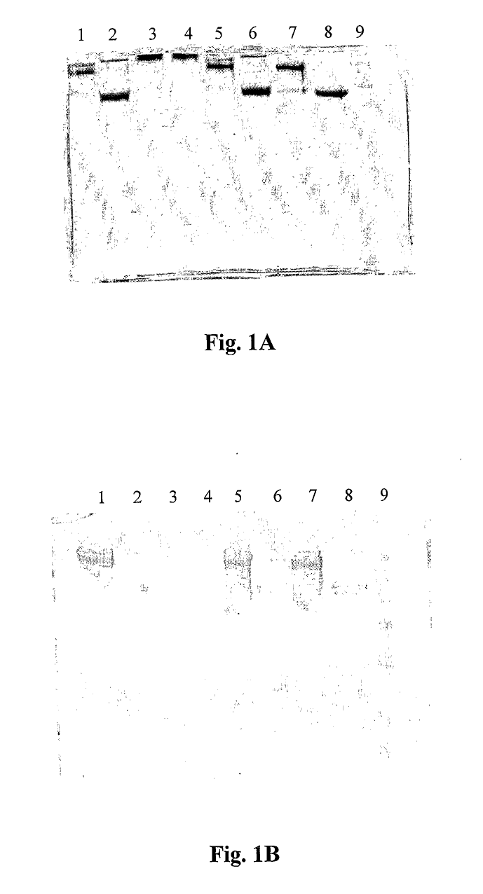

For characterization of the purified coated chicken IgY obtained in (i) to (iii) above, concentration was determined by optical density measurements at 280 nm The protein size was evaluated by 12% SDS-PAGE, followed by staining with Coomassie blue. The results are shown in FIG. 1A. To characterize the antigenicity of the coated chicken IgY molecules, the gels were submitted to Western blotting using anti-IgY antibodies (Sigma-Aldrich). The results are shown in FIG. 1B. In both FIGS. 1A and 1B, lanes 1 and 2 show chicken IgY coated by procedure (iii), non-boiled and boiled, respectively; lanes 3 and 4 show chicken IgY coated by procedure (ii), non-boiled and boiled, respectively; lanes 5 and 6 show chicken IgY coated by procedure (i), non-boiled and boiled, respectively; lanes 7 and 8 show non-coated chicken IgY, non-boiled and boiled, respectively; and lane 9 show molecular weight markers. As shown in FIG. 1B, chicken IgY coated by procedure (ii) is totally masked and is not recognized by anti-IgY antibodies in comparison with chicken IgY coated by procedures (i) or (iii). These results encouraged the inventors to select procedure (ii) for further investigations.

### EXAMPLE 2

### Preparation of Antibody Coated by Mannose and Oleic Acid

In this preliminary example, cow IgG was coated with mannose and oleic acid. D(+)-mannose (10 mg) was added to a 0.5 ml solution of cow IgG (225 μg/ml solution in PBS 50 mM, pH=7.0), the solution was shaken for 12 hours at room temperature, then 3 mg of p-TSA and 9 mg of EDC were added, and the mixing was continued for additional 12 hours. The reaction mixture was dialyzed against 1 liter of PBS solution (50 mM, pH=7.0) three times. To the remaining mixture, 5 μl of oleic acid, 3 mg of p-TSA and 9 mg EDC were added, and the solution was mixed for additional 12 hours.

### EXAMPLE 3

### Masking of Antibody Surface in an Antibody-Antigen Complex

After the preparation of antibody molecules coated by small molecules as described in Examples 1 and 2 above, it was of interest to mask the antibody surface in an antibody-antigen complex according to the method of the present invention. For this purpose, IgY obtained from chicken injected with E. coli was complexed with the whole bacteria as described below.

Heat killed virulent E. coli O78:K80 was injected to chicken (Leghom layers, n=4), twice in two-weeks interval and antibodies were isolated from egg yolk. The purified chicken IgY fraction obtained from the egg yolk was incubated with the virulent E. coli O78:K80 (10⁸) for 2 hours at 37° C. The complex was centrifuged and washed twice in PBS. The pellet containing the complex was suspended in PBS buffer and coated with D(+)-mannose as described in Example 1 (ii). The mannose-coated complex was washed twice following centrifugation, to separate free unattached IgY. Separation between the bacteria and attached masked-antibody was performed by suspending the pellet (containing the bacteria) in glycine buffer (0.1M, pH 2.7) followed by centrifugation (10K) for 5 minutes. The supernatant containing the masked antibody was collected and the pH was adjusted to 7.0 by Tris buffer (2M, pH 9.0).

The antibody masked as described above, when injected, is not expected to induce an immune response. On the other hand, due to protection of the binding site (idiotype) by the antigen during the masking step, the ability to detect and bind to the epitope to which it was attached during the masking step is conserved.

### EXAMPLE 4

### Masking of Antibody Against Soluble Antigen (Snake Toxin)

Soluble antigens such as snake toxins and other soluble antigenic peptides can be attached to polymer beads such as polystyrene beads.

Vaccines based on masked antibodies against snake toxin can be prepared by injecting a snake toxin to chicken as described in Example 3 above. The purified IgY fraction from egg yolk is incubated with the snake toxin attached to microporous polystyrene beads, the complex is centrifuged, and washed. The pellet is suspended and masked with mannose. The complex containing the masked IgY antibody is washed, the masked antibody is separated from the toxin and collected from the supernatant, and the pH is adjusted to 7.0.

The anti-toxin mannose-masked antibody, when injected, should not induce immune response, but should be able to detect and bind to the toxin epitope to which it was attached during the masking step, and thus neutralize the toxin.

### EXAMPLE 5

### Masking of Cholera Toxin and E. coli Enterotoxin

The heat-labile enterotoxin of Escherichia coil (LT) and cholera toxin of Vibrio cholera (CT) cause two very serious diseases in developing countries. Both have similar pathogenic effects and show 95% sequence similarity. Both toxins bind to cellular receptors, GM1 ganglioside, on cell membrane, followed by entrance into the cells whereby the A subunit, upon proteolytic activation. causes diarrhea.

The CT and LT toxins are immunogenic. Decreasing the immunogenicity enables insertion of molecules into cells. This may be especially important for molecules that are administered via oral or skin routes.

### EXAMPLE 6

### Masked Antibody Shows Reduced Antigenicity

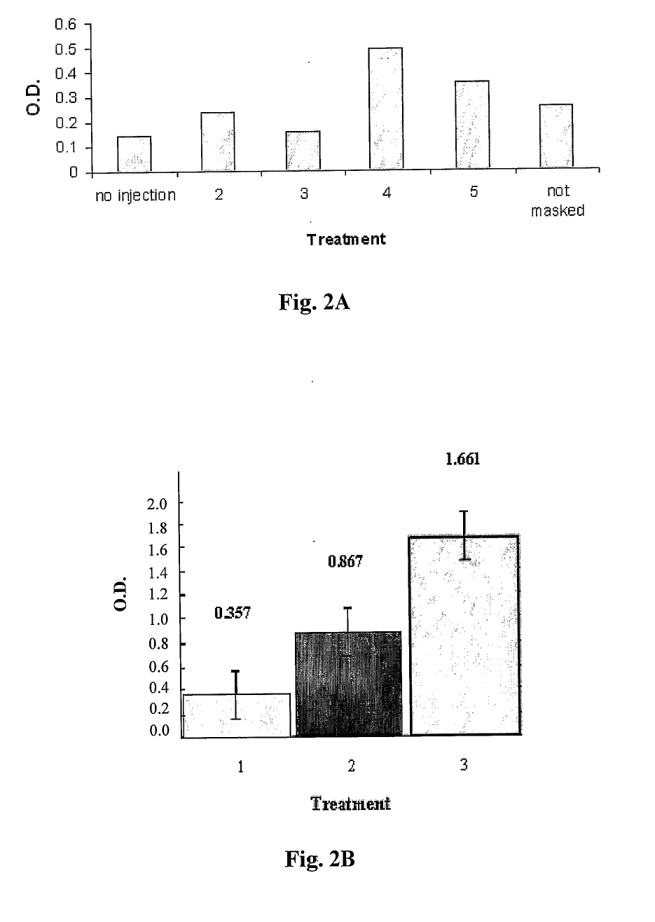

Cow IgG antibodies were coated with mannose and/or oleic acid by the procedures (i), (ii) and (iii) described in Examples 1 and 2 above, and injected twice (50 μg IgG/chicken along with emulsion of Incomplete Freund's Adjuvant) to six chickens. Two weeks later, blood was drawn from the chicken and anti-cow chicken IgY was tested in the chicken serum by ELISA. ELISA plate was coated with cow IgG antibodies, and incubated for two hours at 37° C. with sera of chicken, followed by incubation for two hours at 37° C. with second antibody, rabbit anti-chicken IgY-conjugated to horseradish peroxidase (HRP) (Sigma-Aldrich). The different immunization schedules are summarized in Table 1 and the ELISA results are depicted in FIG. 2A.

TABLE 1
Immunization of chicken with coated cow IgG

| | Cow igG | | | | | |
|---|---|---|---|---|---|---|
| Treatment | Antibody | Mannose | EDC | pTSA | Oleic Acid | Dialysis |
| No injection | − | − | − | − | − | − |
| 2 | + | + | + | + | − | − |
| 3 | + | + | + | + | + | + |
| 4 | + | + | + | + | + | − |
| 5 | + | − | + | + | + | − |
| Not masked | + | − | − | − | − | − |

The results of ELISA titration of anti-cow IgY antibodies found in the chicken sera, depicted in FIG. 2A, show that immunization schedules 2 (cow IgG antibodies coated with mannose+EDC+pTSA) and 3 (cow IgG antibodies coated with mannose+EDC+pTSA+oleic acid and dialysis), significantly reduced the antigenicity of the cow IgG in chicken.

Similar experiments were carried out with human IgG masked with mannose and oleic acid. Chickens (4 chicken/group) were immunized twice at two weeks intervals with human IgG (50 μgG/chicken along with emulsion of Incomplete Freund's Adjuvant), human IgG masked with mannose and oleic acid, or unmasked human IgG. Two weeks following the second immunization, blood was harvested and serum isolated from immunized and non-immunized chicken. Samples of serum were subjected to ELISA specific for the detection of human IgG antibody. The ELISA plates were coated with human IgG (100 ng/well) and the samples of chicken serum containing antibody capable of binding human IgG in the plates were detected with rabbit anti-chicken IgY-conjugated to horseradish peroxidase (HRP). The results of ELISA titration of anti-human IgY antibodies found in the chicken sera, depicted in FIG. 2B, show that human IgG masked with mannose and oleic acid (2) were significantly less immunogenic than unmasked antibody (3).

### EXAMPLE 7

### Masking of Adenoviruses

Viruses are used or proposed as gene therapy vectors and for other purposes. Viruses attach to the target cell by an attachment molecule (AM), which is specific to the virus. Protection of the AM followed by masking of the whole virus enables entrance of the virus to cells while decreasing antibody response.

Egg-drop syndrome (EDS) chicken adenovirus (10^{6.4} EID50/mL) was incubated with hyper-immune sera against the knob part of the fiber protein (adenovirus AM) for 3 hours at 37° C. (the knob part and the antibodies were produced in the inventors' laboratory). The virus-antibody complex was ultracentrifuged for 1 h, at 27000 RPM, at 4° C., followed by two washes and ultracentrifugations to discard free antibody. The pellet was suspended and coated with mannose+EDC+pTSA as described in Example 3. Following two washes, the pellet (containing the coated virus-antibody complex) was collected, the complex was separated by decrease of pH (0.1M glycine, pH 2.7) and coated adenoviruses were isolated from the pellet following centrifugation.

To test the antigenicity of the modified mannose-masked adenovirus, dot blot analysis was conducted using anti-EDS antibodies (produced in the inventors' laboratory), with samples either containing the mannose-masked adenovirus or unmasked adenovirus, as detailed in Table 2. The samples were transferred to nitrocellulose membranes (Amersham) and blocked by milk buffer (1% non-fat milk in PBS). The samples of Groups A and B were incubated with anti-EDS antibodies (diluted 1:500) for 1 hour at 37° C. After several washes in PBS, the membranes were incubated with secondary antibody (rabbit anti-chicken IgG-conjugated to HRP, diluted 1:1000) (Sigma-Aldrich) for 1 hour at 37° C., while samples of Groups C and D (negative control) were incubated only with secondary antibody, followed by incubation in the substrate solution 3,3′ diaminobenzidine (Sigma-Aldrich).

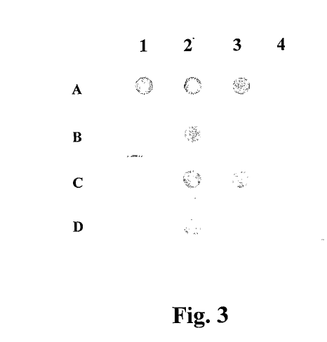

TABLE 2
Samples for dot blot analysis depicted in FIG. 3

| Membrane loading | | | | |
|---|---|---|---|---|
| | Anti- | | | Detection |
| Sample | knob | | | Anti- |
| number | antibody | Masked | | EDS |
| in dot | prior | with | | Antibody |
| blot | masking | mannose | Separation* | on dot-blot |
| A-1 | − | − | − | + |
| A-2 | + | − | − | + |
| A-3 | + | − | + | + |
| A-4 | − | + | − | + |
| B-1 | + | + | − | + |
| B-2 | + | + | + | + |
| B-3 | Irrelevant | + | − | + |
| | Ab* | | | |
| C-1 | − | − | − | − |
| C-2 | + | − | − | − |
| C-3 | + | − | + | − |
| C-4 | − | + | − | − |
| D-1 | + | + | − | − |
| D-2 | + | + | + | − |
| D-3 | Irrelevant | + | − | |
| | Ab* | | | |

*Irelevant Ab-from unvaccinated chicken

The dot blot results, as shown in FIG. 3, revealed that the masking procedure significantly reduces the identification of the masked adenovirus (B1) by the anti-EDS antibody, as compared with unmasked virus (A2). Furthermore, separation of masked virus (B2) allows the anti-EDS antibody to bind to the masked virus and stronger identification is detected as compared with non-separated (B1). A1 serves as a positive control, B3 serves as a negative control (includes virus incubated with an antibody from unvaccinated chicken i.e. irrelevant Ab). C1 is another negative control (includes virus without antibody). Lines C and D were loaded with the same samples as lines A and B, but were incubated only with the secondary antibody.

### EXAMPLE 8

### Agglutination Tests with Mannose-Masked Adenoviruses

Since specific domains on the adenovirus fiber knob were shown to mediate the agglutination of erythrocytes, agglutination tests were performed both following binding of the anti-knob antibodies to the adenovirus and after masking with mannose.

Adenovirus was incubated with anti-knob antibodies for 3 hours at 37° C. The complex was ultracentrifuged for 1 h, at 27000 RPM, 4° C. EDS fiber protein causes agglutination of red blood cells, whereas antibodies against this protein inhibit this hemagglutination. The test was performed against 4 hemagglutination activity (HA) units of EDS virus in a 96-well microtiter plate and expressed as log₂ geometric mean titer. The first agglutination test was performed following the binding of the adenovirus to the anti-knob antibody. The results in Table 3 show that binding of the anti-fiber knob antibody to the adenovirus abolished the virus agglutination power (titer=0).

TABLE 3
Agglutination tests with adenovirus bound to anti-knob antibody

| Sample | Titer |
|---|---|
| Untreated adenovirus | 6 |
| (before binding) | |
| Virus incubated with | 0 |
| anti-knob Ab | |
| Virus incubated with | 1 |
| irrelevant Ab | |
| (from unvaccinated chicken) | |
| Virus after ultracentrifugation | 5 |
| Blood cells (blank) | 0 |

After ultracentrifugation of the virus-anti-knob antibody complex, the pellet containing the complex was suspended and coated with mannose+EDC+pTSA.

To ensure that the glycine buffer used for separation of the mannose-masked virus from the virus-antibody complex does not interfere with the adenovirus agglutination power, a second agglutination test was performed after separation of the mannose-masked adenovirus from the masked virus-antibody complex. The results of the second agglutination test are shown in Table 4.

TABLE 4
Agglutination test with mannose-masked adenovirus

| | Time of | |
|---|---|---|
| Sample | elution | Titer |
| Virus incubated with Ab | 2 min | 13 |
| Virus incubated with Ab | 1 hr | 13 |
| Glycine Buffer | — | 0 |
| Untreated Virus | — | 7 |
| Blood cells (Blank) | — | 0 |

In similar experiments, we found that masking of EDS chicken adenovirus with mannose in the absence of antibodies specific for the attachment molecule of the virus (AM) resulted in lack of agglutination (not shown). However, the agglutination activity remained unchanged using virus which was complexed with either anti-EDS antibodies (comprising general+specific antibodies to AM) or AM specific antibodies prior to mannose masking and then was separated from the antibodies after masking.

In all, the results obtained show that mannose-masked adenovirus, which maintain the agglutination activity, can be obtained by protecting the attachment molecule (AM) in the virus prior to mannose masking with AM specific antibodies and by separating the antibodies from the virus after mannose masking.

### REFERENCES

Kobayashi, K., Hirano, A., Ohta, A., Yoshida, T., Takahashi, K. and Hattori, M. (2001) Reduced immunogenicity of beta-lactoglobulin by conjugation with carboxymethyl dextran differing in molecular weight. J Agric Food Chem. 49, 823-831.

Koumenis I L, Shahrokh Z, Leong S, Hsei V, Deforge L, and Zapata G. (2000) Modulating pharmacokinetics of an anti-interleukin-8 F(ab′)(2) by amine-specific PEGylation with preserved bioactivity. Int J Pharm. 30,198(1):83-95.

Mehvar, R. (2003) Recent trends in use of polysaccharides for improved delivery of therapeutic agents: pharmacokinetic and pharmacodynamic perspectives. Curr Pharm Biothechnol. 4, 283-302.

O'Riordan, C. R., Lachapelle, A., Delgado, C., Parkes, V., Wadsworth, S. C., Smith, A. E. and Francis, G. E. (1999) PEGylation of adenovirus with retention of infectivity and protection from neutralizing antibody in vitro and in vivo. Hum Gene Ther. 10, 1349-1358.

Pedley R B, Boden J A, Boden R, Begent R H, Turner A, Haines A M, King D J. (1994) The potential for enhanced tumour localisation by poly (ethylene glycol) modification of anti-CEA antibody. Br. J. Cancer, 70: 1126-1130

1. A method for obtaining a modified protein or virus with an intact native binding site and decreased antigenicity, which comprises masking with non-immunogenic molecules the protein or the virus surface, except for the protein or the virus binding site.

2. The method of claim 1, wherein said non-immunogenic molecules are small molecules.

3. The method of claim 2, wherein said non-immunogenic molecules are monosaccharides or fatty acids, or both.

4. The method of claim 3, wherein said monosaccharide is a C₃-C₆ ketose or aldose.

5. The method of claim 3, wherein said fatty acids are saturated or unsaturated C₃-C₂₀ fatty acids.

6. The method of claim 5, wherein said fatty acid is a saturated or unsaturated C₃-C₂₀ fatty acid.

7. The method of claim 3, wherein said non-immunogenic molecules are mannose and oleic acid.

8. The method of claim 1, wherein said non-immunogenic molecules are polymers or a polysaccharide.

9. The method of claim 1, wherein molecules which target specific cells or tissues such as cancerous cells or tissues, are chemically attached to functional groups of said non-immunogenic molecules.

10. The method of claim 1, wherein said modified protein is a modified antibody, said method comprising:
(i) attaching an antigen recognized by said antibody to a surface;
(ii) incubating the antibody with said attached antigen, thus forming an antigen-antibody complex;
(iii) masking the antibody surface by chemically attaching non-immunogenic molecules to the antibody surface in the antigen-antibody complex, thus obtaining an antigen-masked antibody complex; and
(iv) separating the masked antibody from the antigen, thus obtaining the desired modified antibody masked with the non-immunogenic molecule, said modified antibody exhibiting an intact native binding site and decreased antigenicity as compared with the unmodified antibody.

11. A modified antibody obtainable by the method of claim 10, said antibody being biologically active, having its native binding site intact and exhibiting decreased immunogenicity as compared with the unmodified antibody.

12. A modified antibody wherein its surface, except for the native binding site, is masked with small molecules covalently linked to functional groups of the antibody molecule, and said small molecules are monosaccharides or fatty acids, or both.

13. The modified antibody of claim 12, wherein said monosaccharide is mannose and said fatty acid is a saturated or unsaturated C₃-C₂₀ fatty acid.

14. The modified antibody of claim 11, wherein said antibody is a monoclonal antibody.

15. The modified antibody of claim 11, wherein said antibody is a polyclonal antibody.

16. The modified antibody of claim 11, wherein said antibody is not antigenic within the same or heterologous species.

17. The modified antibody of claim 11, wherein the antibody is an anti-tumor antibody.

18. The method of claim 1, wherein said modified virus is a modified replication-defective virus, said binding site is a native knob binding site, and said method comprises the steps:
(i) incubating the replication-defective virus with an antibody to the knob binding site, thus forming a virus-antibody complex;
(ii) masking the virus surface by chemically attaching non-immunogenic molecules to the virus surface in the virus-antibody complex, thus obtaining a masked virus-antibody complex;
(iii) separating the masked virus from the antibody; and
(iv) removing the residual antibody from the solution by centrifugation,
thus obtaining the desired modified replication-defective virus with an intact native knob binding site and decreased antigenicity as compared with the unmodified virus.

19. A modified replication-defective virus obtainable by the method of claim 18, said virus having a native knob binding site and exhibiting decreased immunogenicity as compared with the unmodified virus.

20. A modified replication-defective virus wherein its surface, except for the native knob binding site, is masked with small molecules covalently linked to functional groups of the virus surface, and said small molecules are monosaccharides or fatty acids, or both.

21. The modified virus of claim 20, wherein said monosaccharide is mannose and said fatty acid is a saturated or unsaturated C₃-C₂₀ fatty acid.

22. The modified replication-defective virus of claim 19 selected from an adenovirus, preferably Ad5, a retrovirus, or a recombinant virus which expresses a transgene such as a therapeutic gene for use in gene therapy.

23. A method of claim 1, wherein said modified protein is a modified hormone, said binding site is a receptor binding site, and said method comprises the steps of:
(i) incubating a hormone with its receptor, thus forming a hormone-receptor complex;
(ii) masking the hormone surface by chemically attaching non-immunogenic molecules to the hormone surface in the hormone-receptor complex, thus obtaining a masked hormone-receptor complex; and
(iii) separating the masked hormone from the receptor;
thus obtaining the desired modified masked hormone with an intact receptor binding site and decreased antigenicity as compared with the unmodified hormone.

24. A modified hormone obtainable by the method of claim 23, said hormone having a native hormone binding site and exhibiting decreased immunogenicity as compared to the unmodified hormone.

25. A method of claim 1, wherein said modified protein is a modified enterotoxin, said binding site is a receptor binding site, and said method comprises the steps of:
(i) incubating an enterotoxin with its receptor, thus forming an enterotoxin-receptor complex;
(ii) masking the enterotoxin surface by chemically attaching non-immunogenic molecules to the enterotoxin surface in the enterotoxin-receptor complex, thus obtaining a masked enterotoxin-receptor complex; and
(iii) separating the masked enterotoxin from the receptor; thus obtaining the desired modified masked enterotoxin with an intact receptor binding site and decreased antigenicity as compared with the unmodified enterotoxin.

26. A modified enterotoxin obtainable by the method of claim 25, said enterotoxin having a native hormone binding site and exhibiting decreased immunogenicity as compared to the unmodified enterotoxin.

27. The modified enterotoxin of claim 26, wherein said modified enterotoxin is the enterotoxin of Escherichia coli (LT) or the cholera toxin of Vibrio cholera (CT) with an intact GM1 ganglioside receptor-binding site.

28. The method of claim 4 wherein said monosaccharide is a C₅-C₆ aldose.

29. The method of claim 28 wherein said C₅-C₆ aldose is mannose.

30. The method of claim 6, wherein said saturated C₃-C₂₀ fatty acid is capric acid, lauric acid, myristic acid, palmitic acid, stearic acid, or arachidic acid, and said unsaturated C₃-C₂₀ fatty acid is palmitoleic acid, oleic acid, vaccenic acid, linoleic acid, γ-linolenic acid, linolenic acid, or arachidonic acid.

31. The method of claim 8, wherein said polymer is a hydroxylated polyether and said polysaccharide is dextran, modified dextran, pullulan or mannan.

32. The method of claim 31 wherein said polymer is polyethylene glycol (PEG).

33. The modified antibody of claim 13, wherein said unsaturated C₃-C₂₀ fatty acid is oleic acid.

34. The modified virus of claim 21 wherein said unsaturated C₃-C₂₀ fatty acid is oleic acid.
